(19) United States
(12) Patent Application Publication
(10) Pub. No.: US 2012/0109111 A1
(43) Pub. Date: May 3, 2012

(54) FENESTRATION SYSTEM

(75) Inventor: Jiangming LI, Shijiazhuang (CN)

(73) Assignee: EMPIRE TECHNOLOGY DEVELOPMENT LLC, Wilmington, DE (US)

(21) Appl. No.: 12/915,772

(22) Filed: Oct. 29, 2010

(51) Int. Cl. A61M 27/00

(52) U.S. Cl. 604/543

(57) ABSTRACT

A fenestration system can be useful for draining cysts, abscesses, or other bodily cavities. A fenestration system can include: a drainage screwdriver; a drainage screw removably receivable onto the drainage screwdriver; and a drainage cap removably coupleable with the drainage screw when the drainage screw removed from the drainage screwdriver. The drainage screwdriver can receive the drainage screw so that the drainage screw can be screwed through tissue and/or cyst or abscess sac. The drainage screwdriver can then release the drainage screw so that it is inserted into the tissue, cyst, or abscess so as to be capable of drainage. A lumen in the drainage screw can function similarly to a drainage tube. The drainage cap can be applied to the drainage screw to close the lumen to inhibit other fluids or particles from entering into the tissue, cyst, or abscess via the lumen.

### BACKGROUND

Cysts may occur under the skin or mucosa and result in the formation of a sac that has a distinct membrane from the nearby or surrounding tissue. The cyst sac may contain air, fluids, or semi-solid biological materials that are not considered pus. When pus is present, a sac or cavity may be considered an abscess. Once formed, a cyst may be surgically removed. In many instances cysts cause physiological symptoms such as pain, itch, or general discomfort. Abscesses may also require surgical intervention. In both cysts and abscesses, it may be important to penetrate into the sac and drain the contents of the sac. However, the drainage may be slow and/or may require a drainage tube or other drainage feature to be continually inserted into the sac until the contents are drained. Also, a drainage tube may fall out from the sac unless properly secured. Additionally, tissues, fluids, and other materials may drop into the cyst, and removal of such materials may be difficult. Moreover, attempted drainage may be unsuccessful, and may lead to infection.

When a cyst or abscess is present in a mouth, the complications may arise during treatment or drainage due to the moist environment and the presence of food particles. Such food particles are a nuisance for treating an osteocyst of the jaw and often lead to infection. A similar problem occurs in abscesses of the mouth.

In view of the foregoing, there remains a need for improved tools and techniques for draining a cyst or abscess, especially in the mouth.

### SUMMARY

In one embodiment, a fenestration system can be useful for draining cysts, abscesses, or other bodily cavities. A fenestration system can include: a drainage screwdriver; a drainage screw removably receivable onto the drainage screwdriver; and a drainage cap removably coupleable with the drainage screw when the drainage screw removed from the drainage screwdriver. The drainage screwdriver (i.e., fenestration screwdriver), can receive the drainage screw (i.e., fenestration screw) so that the drainage screw can be screwed through tissue and/or cyst or abscess sac. The drainage screwdriver can then release the drainage screw so that it is inserted into the tissue, cyst, or abscess so as to be capable of drainage. A lumen in the drainage screw can function similarly to a drainage tube by the drainage screw having a length to traverse the tissue and into the sac so the contents can drain. The drainage cap can be applied to the drainage screw to close the lumen to inhibit other fluids or particles from entering into the tissue, cyst, or abscess via the lumen.

In one embodiment, a fenestration screwdriver can include: an elongate shaft having drainage screw engaging members configured to engagedly receive a drainage screw; a sharp tip at one end of the elongate shaft; a handle at one end of the elongate shaft opposite of the sharp tip; and a drainage screw release system configured to retain the drainage screw during implantation and to release the drainage screw from the elongate shaft after implantation.

In one embodiment, a fenestration screw can include a substantially tubular body or screw-shape body having an outer surface and an inner surface. The outer surface can include: a distal end having a first opening to an internal lumen; a screw section proximal to the distal end and including screw threads; a tissue stop proximal to the screw section and having a larger cross-sectional profile compared to the screw section and configured to inhibit the fenestration screw from advancing further into a tissue past the tissue stop; a releasable member proximal to the tissue stop and being configured to be coupled to a release system during implantation and be released from the release system after implantation, wherein the releasable member is also configured to removably receive a screw cap; and a proximal end having a second opening to the internal lumen. The inner surface can include: a smooth luminal surface proximal of the first opening and defining the internal lumen; and one or more engagement members formed into the smooth luminal surface and configured to engage with a fenestration screwdriver. The fenestration screw may also include a removable fenestration cap for use in closing the lumen of the fenestration screw.

In one embodiment, a fenestration screw cap can include: a flexible, circular sheet having a perimeter surface; and a flexible ring coupled with the perimeter surface of the circular sheet. The flexible ring may be substituted with a threaded member that can be screwed onto the drainage screw when the drainage screw includes complementary threading. The circular sheet is shaped to fit over a circular lumen of a drainage screw; however, if the lumen has another shape, the flexible sheet can have a different shape other then circular.

In one embodiment, the fenestration system can be used in a method of draining a fluid from a subject. Such a method can include: piercing a tissue associated with the fluid with a tip of a drainage screwdriver; driving a drainage screw into the tissue using a drainage screwdriver that is removably coupled to the drainage screw such that the drainage screw is inserted through the tissue and contacts the fluid; and uncoupling the drainage screwdriver from the drainage screw such that the drainage screw is retained in the tissue, and wherein an internal lumen of the drainage screw drains the fluid from the tissue.

In one embodiment, a fenestration system can be used in a method of draining an oral cyst from a subject. The method can include: piercing a cyst sac in a jaw tissue with a sharp tip of the drainage screwdriver; driving the drainage screwdriver such that the drainage screw is inserted through the cyst sack and contacts cyst fluid located therein; removing the drainage screwdriver from the drainage screw such that the drainage screw is retained in the cyst sack with an internal lumen of the drainage screw drains the cyst fluid from the cyst sack; and applying the drainage cap to the drainage screw so as to seal the internal lumen of the drainage screw.

In one embodiment, a fenestration screwdriver can include: an elongate shaft having an internal lumen defined by an inner surface; a screw shaft located within the internal lumen of the elongate shaft such that a distal end and a proximal end each extend from the internal lumen; an elongatable/retractable member having a sharp tip and an outer surface movably coupled with the inner surface of the elongate shaft so as to be at least partially located within the internal lumen of the elongate shaft and having an threaded inner surface defining an inner lumen of the elongatable/retractable member, the threaded inner surface is rotatably coupled with the distal end screw shaft that such that the screw shaft is at least partially located within the internal lumen of the elongatable/retractable member; a handle having an internal lumen defined by an inner surface, the proximal end of the screw shaft extending through the internal lumen of the handle; and a screw knob coupled to the screw shaft such that rotation of the screw knob rotates the screw shaft.

In one embodiment, the fenestration screwdriver can include a limit screw located within the handle and having an internal lumen defined by a threaded inner surface that is rotatably coupled with the screw shaft.

In one embodiment, the fenestration screwdriver can include a gasket associated with the limit screw and having an internal lumen defined by an inner surface, where the screw shaft passes through the internal lumen.

In one embodiment, the fenestration screwdriver can include one or more engaging members located on an outer surface of the elongate shaft, wherein the engaging members are configured to engage with a drainage screw.

In one embodiment, a fenestration system can include a fenestration screwdriver that includes an elongatable/retractable member as described herein and a drainage screw having an internal lumen. The fenestration system can also include a drainage screw cap.

In one embodiment, a fenestration method can include: providing a fenestration system as described herein that has a fenestration screwdriver with a elongatable/retractable member; piercing a tissue with the sharp tip of the elongatable/retractable member; retracting the elongatable/retractable member into the drainage screw; screwing the drainage screw into the tissue; and withdrawing the elongatable/retractable member from the lumen of the drainage screw.

In one embodiment, the method includes elongating the elongatable/retractable member to pierce the tissue.

In one embodiment, the method includes placing the drainage screw on the fenestration screwdriver.

In one embodiment, the method includes a practitioner, such as a doctor or dentist, holding the handle of the fenestration screw driver during the piercing, retracting, and/or screwing.

In one embodiment, the method includes rotating the screw knob to perform the piercing and/or retracting.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1A is a perspective view of an illustrative embodiment of a fenestration system.

FIG. 1B is a side view of the fenestration system of FIG. 1A.

FIG. 1C is a perspective view of an illustrative embodiment of a fenestration screwdriver of the fenestration system of FIGS. 1A-1B.

FIG. 1D is a side view of an illustrative embodiment of a fenestration screw of the fenestration system of FIGS. 1A-1B.

FIG. 1E is a cutaway side view of the fenestration screw of FIG. 1D.

FIG. 1F is a top view of the fenestration screw of FIG. 1D.

FIG. 1G is a top view of a screw cap that can be removably received onto the fenestration screw of FIGS. 1D-1F.

FIG. 1H is a side view of the screw cap of FIG. 1G.

FIG. 2A is an illustrative diagram of the fenestration system of FIGS. 1A-1B inserting the fenestration screw of FIG. 1D into a cyst.

FIG. 2B is an illustrative diagram showing the fenestration screw of FIGS. 1D-1F draining the cyst of FIG. 2A.

FIG. 2C is an illustrative diagram showing the screw cap of FIGS. 1G-1H closing the lumen of the drainage screw of FIGS. 1D-1F.

FIGS. 3A-3D include illustrative diagrams showing an embodiment of a fenestration screw driver.

### DETAILED DESCRIPTION

Generally, a fenestration system may be used in medical procedures for draining cysts, abscesses, or other bodily cavities. An example of a fenestration system may include a drainage screwdriver, a drainage screw, and a cap that fits onto the drainage screw. The drainage screwdriver may be configured similarly to an ordinary screwdriver except that it also may include a tissue-piercing tip, a shaft that receives a drainage screw, engagement members that engage with the drainage screw and allow the drainage screw to be screwed into a cyst, and a release member that can selectively release the drainage screw from the screwdriver. The drainage screw may include an internal lumen that extends from an opening in one end to an opening in the other end. The internal lumen may function as a drainage tube when the drainage screw is screwed into a cyst. The cap, which is referred to herein as a drainage screw cap, may be configured to be removably coupleable with the drainage screw so as to close the external access to the internal lumen of the drainage screw when the cap is coupled to the drainage screw, and to be selectively removed when desired.

The drainage screwdriver (i.e., fenestration screwdriver) can receive the drainage screw (i.e., fenestration screw) so that the drainage screw can be screwed through tissue and/or cyst or abscess sac. The drainage screwdriver can be operated to activate the release member so as to release the drainage screw so that it is implanted into the tissue, cyst, or abscess so as to be capable of drainage through the lumen. The lumen in the drainage screw can function similarly to a drainage tube, and the threading of the drainage screw can retain the drainage screw in the tissue, cyst, or abscess. The drainage cap can be applied to the drainage screw to close the lumen to inhibit other fluids or particles from entering into the tissue, cyst, or abscess via the lumen.

### Fenestration System

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All embodiments and components and features illustrated in the figures and/or described herein can be applied to other embodiments of the fenestration system, fenestration components, and methods of using the same.

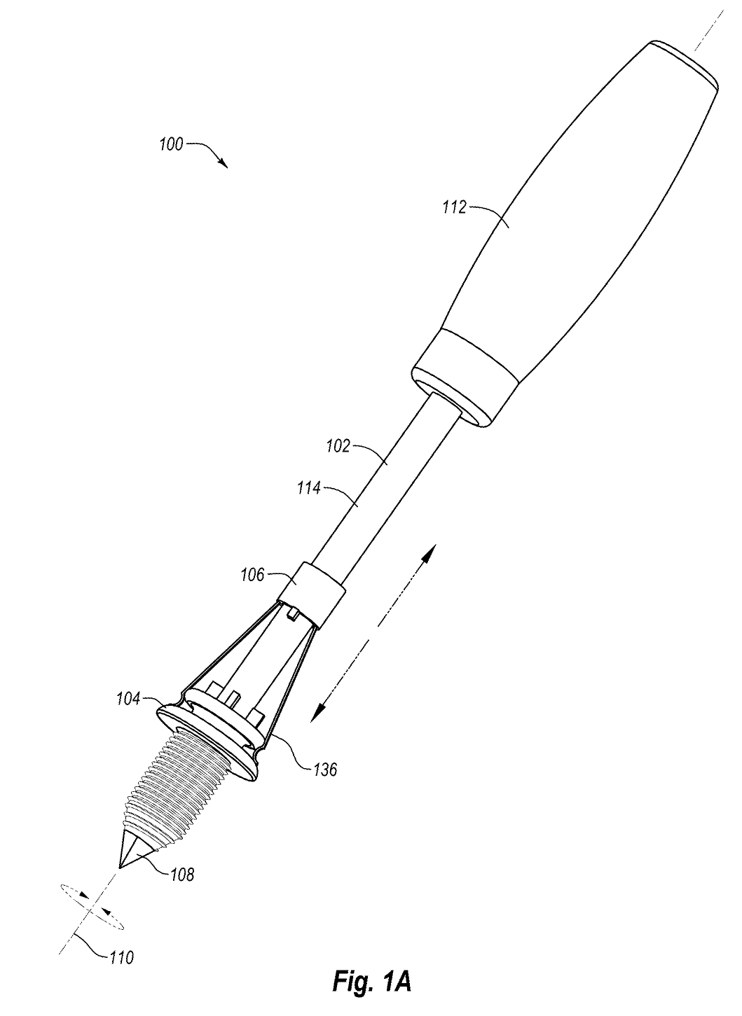

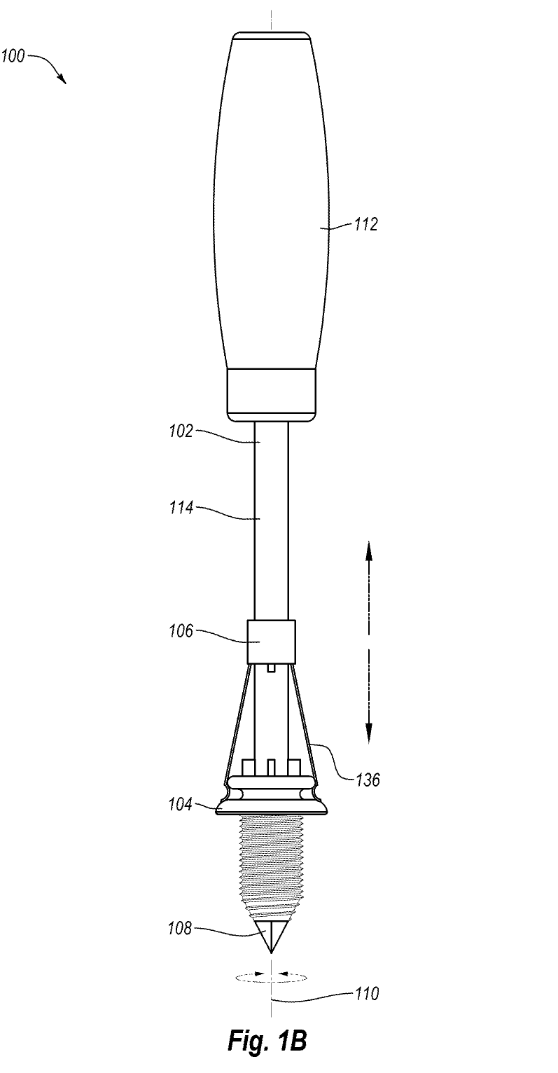

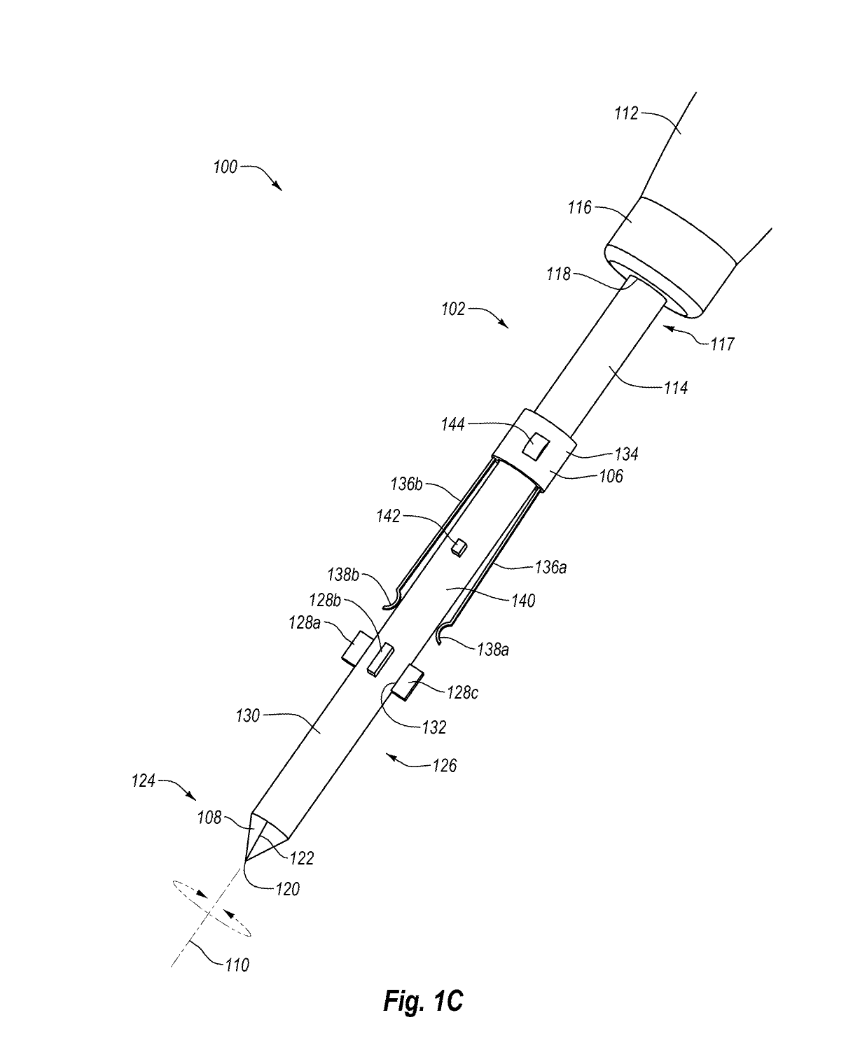

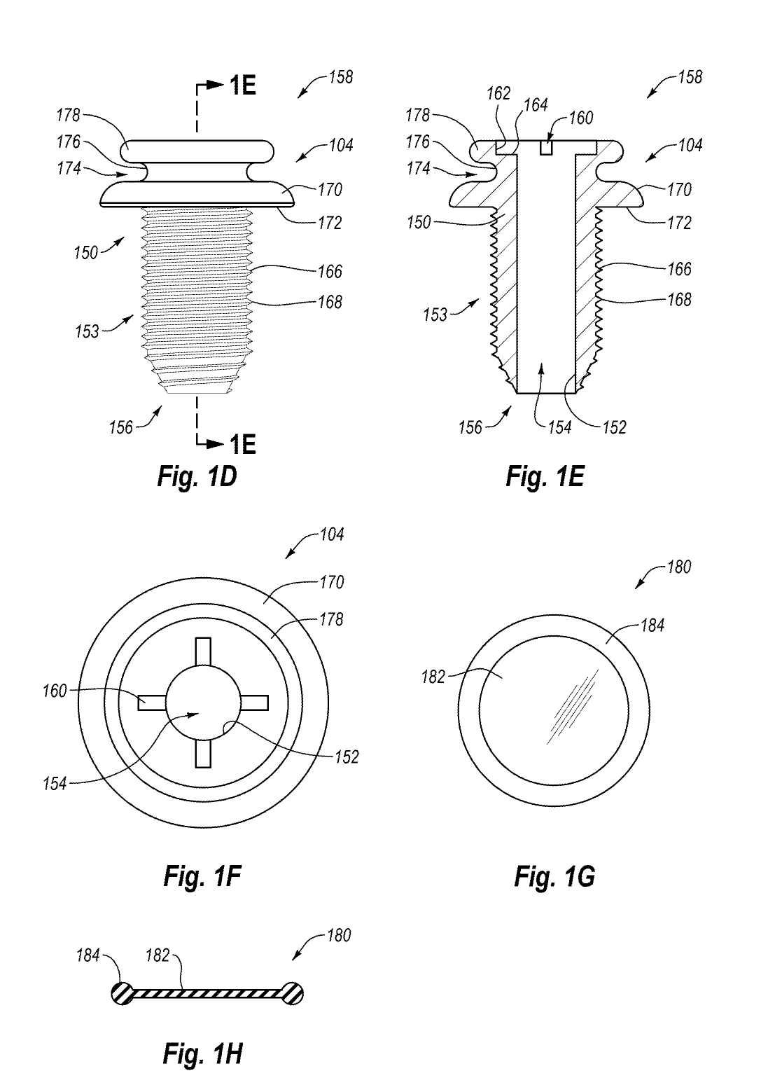

FIGS. 1A-1B show a fenestration system 100 that includes a drainage screwdriver 102 retaining a drainage screw 104 in a manner that allows for the drainage screwdriver 102 to screw the drainage screw 104 into a tissue. The drainage screwdriver 102 includes a distal tip 108 that is sharp and configured for piercing a tissue. The distal tip 108 also indicates the axis of rotation 110 for the drainage screwdriver 102. An elongate shaft 114 couples the distal tip 108 to an opposite handle 112. The drainage screwdriver 102 includes a release member 106 that is slidably coupled with the elongate shaft 114 as shown by the arrows. The release member 106 includes one or more screw clips 136 that are each configured to retain the drainage screw 104 on the drainage screwdriver 102 during insertion of the drainage screw 104 into a tissue, and configured to release the drainage screw 104 when the release member 106 is activated. Activation of the release member 106 can be performed by sliding the release member 106 away from the drainage screw 104 so that the screw clips 136 release the drainage screw 104.

FIG. 1C shows the drainage screwdriver 102 of the fenestration system 100 of FIGS. 1A-1B. The drainage screwdriver 102 is shown without the drainage screw 104. As shown, the drainage screwdriver 102 includes a distal tip 108 coupled to an elongate shaft 114, which in turn is coupled to the handle 112. The distal tip 108 is included at a tissue piercing end 124 of the drainage screwdriver 102 that has a sharp tip 120 that is sufficiently sharp for penetrating a tissue upon contact and application of slight pressure. Also, the distal tip 108 can include a blade 122 that can facilitate piercing a tissue and forming an incision in the tissue of a size sufficient for receiving the drainage screw 104. The blade 122 can be at any angle from about 0 degrees to about 90 degrees with respect to the axis of rotation 110. The blade 122 may only traverse a portion of the distal tip 108 or it can extend all the way to the elongate shaft 114.

The distal portion 126 (e.g., drainage screw receiving segment) of the elongate shaft 114 is configured with a smooth surface 130 that allows for the drainage screw 104 to slide onto and off from the elongate shaft 114. The smooth surface 130 can extend from the distal tip 108 to the handle 112; however, the elongate shaft 114 can include one or more screw engaging members 128a-128c which may or may not be smooth. These screw engaging members 128a-128c are configured for engaging with the drainage screw 104 so that rotation of the drainage screwdriver 102 rotates the drainage screw 104 and pressure applied to the screwdriver is transferred to the drainage screw 104. While the engaging members 128a-128c are shown as protrusions from the elongate shaft 114, they can be indentations, a roughened surface, or the like. The engaging members 128a-128c can have substantially any shape that allows for the described functionality. When implemented as protrusions, the engaging members 128a-128c can be integrated with the elongate shaft 114 or received into slots 132 in the elongate shaft 114.

The elongate shaft 114 also includes a release member surface 140 that slidably receives the release member 106 proximal of the engaging members 128a-128c. As such, the release member surface 140 can be smooth and have reduced friction, as can the inner surface of the release member 106 that slides over the release member surface 140. The release member 106 is configured to retain the drainage screw 104 on the elongate shaft 114 while the drainage screwdriver 102 is rotated so as to screw the drainage screw 104 into a tissue. In order to retain the drainage screw 104 on the elongate shaft 114, the release member 106 includes one or more screw clips 136a-136b. The screw clips 136a-136b extend from a collar 134 of the release member 106 distally toward the distal tip 108. The collar 134 is the member that slides over the release member surface 140 and which can have the smooth inner surface. Each screw clip 136a-136b includes a clip feature 138a-138b that engages with the drainage screw 104 such that activation of the release member 106, such as by sliding the collar 134 distally, releases the drainage screw 104 from these clip features 138a-138b. The elongate shaft 114 also includes a collar stop 142 that is distal of the collar 134 and configured to stop the collar 134 from advancing distally over the elongate shaft 114.

Optionally, the release member 106 can include an activation member 144 that is located on or associated with the collar 134. Activation of the activation member 144 can release the drainage screw from the elongate shaft 114 by moving the clip features 138a-138b outwardly or laterally with respect to the axis of rotation 110. Such outward or lateral movement of the clip features 138a-138b releases the drainage screw 104 so that the collar 134 can be retracted proximally. The activation member 114 can be a button, knob, toggle, switch, or other activation mechanism.

The proximal end 117 of the elongate shaft 114 can be coupled to the handle 112 by any way a standard screwdriver shaft is coupled to its handle. As shown, the proximal end 117 of the elongate shaft 114 is received into a handle recess 118 within the handle 112 and a handle fastener 116 fastens the handle 112 to the elongate shaft 114. The fastener 116 shown in FIG. 1C is configured as a friction fastener; however, other types of fasteners can be used, such as screws, bolts, hex keys, adhesive, or others.

FIGS. 1D-1F show an embodiment of a drainage screw 104, where FIG. 1D is a side view, FIG. 1E is a cut-away side view, and FIG. 1F is a top view. As shown, the drainage screw 104 includes a screw body 150 having a luminal surface 152 and an outer surface 153. The luminal surface 152 defines a lumen 154 that passes from a tissue penetrating end 156 to a drainage end 158 such that material from a cyst can pass into the tissue penetrating end 156, through the lumen 154 and out from the drainage end 158. The drainage end 158 of the luminal surface 152 includes one or more engaging member receivers 160 that are configured to receive the engaging members 128 of the elongate shaft 114. As such, each of the engaging member receivers 160 can include one or more receiver walls 162 and one or more receiver stops 164 that cooperate to engage the engaging members 128 so that force applied to the handle 112 is transferred to the one or more engaging members 160 in order to screw the drainage screw 104 into a tissue.

The outer surface 153 can include a threaded surface 166 that includes one or more threads 168. The threaded surface 166 and threads 168 can be configured as any common threaded surface of a screw, bolt, or the like. At the proximal end of the threaded surface 166, the drainage screw 104 includes a tissue stop 170 having a tissue stop surface 172 that is configured to inhibit the drainage screw 104 from being screwed further into a tissue. The tissue stop surface 172 is shown to be about 90 degrees with respect to the threaded surface 166; however, it can range from about 30 degrees to about 90 degrees, from 45 degrees to about 90 degrees, or from about 60 degrees to about 90 degrees, in an obtuse or acute angle orientation.

Additionally, the drainage screw 104 can include a groove 174 that is proximal from the tissue stop 170. The groove 174 is configured to receive the clip features 138a-138b of the release member 106. The groove 174 is also configured to receive the drainage screw cap 180 (FIGS. 1G-1H). The groove 174 is defined at least partially by a groove surface 176. In some instances the groove 174 can be distal to the tissue stop 170.

The drainage end 158 of the drainage screw 104 can include a cap receiver 178 that is configured to receive the drainage screw cap 180 thereover. The cap receiver 178 shown in FIGS. 1D-1E is in the form of a lip or protrusion such that the drainage screw cap 180 is received thereover and fit in the groove 174. While not shown, the cap receiver 178 may be threaded when the cap 180 is threaded.

FIG. 1G is a top view of a screw cap 180 and FIG. 1H is a side view of the screw cap 180 that can be removably received onto the fenestration screw of FIGS. 1D-1F. The screw cap 180 includes a substantially flat cap body 182 that is configured to close off the lumen 154 of the drainage screw 104. A cap ring 184 encompasses the outer perimeter of the cap body 182. The cap ring 184 is configured to be resiliently flexible similarly to an O-ring.

In one embodiment, a fenestration system can be useful for draining cysts, abscesses, or other bodily cavities. A fenestration system can include: a drainage screwdriver; a drainage screw removably receivable onto the drainage screwdriver; and a drainage cap removably coupleable with the drainage screw when removed from the drainage screwdriver. The drainage screwdriver, which can be referred to as a fenestration screwdriver, can receive the drainage screw (i.e., fenestration screw) so that the drainage screw can be screwed through tissue and/or cyst or abscess sac. The drainage screwdriver can then release the drainage screw so that it is inserted into the tissue, cyst, or abscess so as to be capable of drainage. A lumen in the drainage screw can function similarly to a drainage tube. The drainage cap can be applied to the drainage screw to close the lumen to inhibit other fluids or particles from entering into the tissue, cyst, or abscess via the lumen.

In one embodiment, a fenestration screwdriver can include: an elongate shaft having drainage screw engaging members configured to receive a drainage screw; a sharp tip at one end of the elongate shaft; a handle at one end of the elongate shaft opposite of the sharp tip; and a drainage screw release system configured to retain the drainage screw during implantation and to release the drainage screw from the elongate shaft after implantation.

In one embodiment, a fenestration screw can include a tubular body having an outer surface and an inner surface. The outer surface can include: a distal end having a first opening to an internal lumen; a screw section proximal to the distal end and including screw threads; a tissue stop proximal to the screw section and having a larger cross-sectional profile compared to the screw section and configured to inhibit the fenestration screw from advancing further into a tissue past the tissue stop; a releasable member proximal to the tissue stop and being configured to be coupled to a release system during implantation and be released from the release system after implantation, wherein the releasable member is also configured to removably receive a screw cap; and a proximal end having a second opening to the internal lumen. The inner surface can include: a smooth luminal surface proximal of the first opening and defining the internal lumen; and one or more engagement members formed into the smooth luminal surface and configured to engage with a fenestration screwdriver. The fenestration screw may also include a removable fenestration cap for use in closing the lumen of the fenestration screw.

In one embodiment, a fenestration screw cap can include: a flexible, circular sheet having a perimeter surface; and a flexible ring coupled with the perimeter surface of the circular sheet. The flexible ring may be substituted with a threaded member that can be screwed onto the drainage screw when the drainage screw includes complementary threading.

The drainage screwdriver can include a shaft configured to receive the drainage screw thereover. The shaft configuration can be modulated with respect to surface smoothness, shaft length, shaft cross-sectional profile, shaft diameter, or other shaft features. A portion of the shaft can be configured as a drainage screw receiving surface and can be relatively smooth so that the drainage screw can freely slide with respect to the shaft. The drainages crew receiving surface can also include a lubricious coating, such as polyethyleneglycol, lubricant, or other. The inner lumen of the drainage screw can also be smooth and/or include a lubricious coating.

The shaft of the drainage screwdriver also includes one or more drainage screw engaging members. The engaging members can be distal, medial, or proximal with respect to the shaft such that they sufficiently engage the drainage screw. In one option, the drainage screw engaging members can be integral with the shaft. In an alternate option, the screw engaging members can be coupled with the shaft. When coupled with the shaft, the screw engaging members can be received into slots of the shaft. Also, the screw engaging members can be affixed to the shaft by adhesive, welding, brazing, or other coupling.

When multiple screw engaging members are included on the shaft, they can be located equally around the shaft. Alternatively, the screw engaging members can be randomly or unequally located around the shaft.

The shaft can include a body that is solid or hollow, and with a cross-sectional profile that is circle, oval, triangle, square, rectangle, polygon, or derivative thereof, or combination thereof.

The drainage screwdriver may or may not include a handle. When included, the handle can be integral with the shaft so as to be a unitary piece. Alternatively, the handle can be a separate member that is coupled with the shaft. The handle can include a cross-sectional profile that is circle, oval, triangle, square, rectangle, polygon, or derivative thereof, or combination thereof, which profile can be the same or different from the shaft.

In one embodiment, the handle can include one or more grips members that can be configured to be gripped by a practitioner. The surface of the grip has one or more griping members that can provide increased grip to the practitioner. In one option, the grip is ergonomically shaped, such as having indentations to receive fingers and an indentation for receiving the palm and/or thumb.

In instances the handle is coupled to the shaft, the handle can include a recess or aperture configured to receive the shaft therein. Also, the handle can include a fastener that couples the handle with the shaft.

The distal end of the shaft can be coupled to or integrally formed with a sharp tip. The tip can include a tissue piercing tip with sharpness sufficient for piercing tissue upon contact with slight pressure. Also, the tip can include a blade that is sharp and that can facilitate piercing and cutting the tissue. The blade can be smooth, curved, serrated, or other common blade feature. Also, more than one blade can be included, with the plurality of blades being the same or having different configuration.

In one embodiment, the tip can be configured as a screw-type tip that includes threads so that the tip can be threaded through a tissue. The threads can be configured to match with the threads of the drainage screw. Also, the threads can be configured as blades to cut the tissue while being screwed through the tissue.

The tip has a cross-sectional size to permit the drainage screw to pass thereover. As such, the widest portion of the tip can be the same size or smaller than the lumen of the drainage screw and/or the shaft.

The drainage screwdriver includes a drainage screw release member that is configured to retain the screw during implantation and to release the screw after being sufficiently implanted. The drainage screw release member can include one or more drainage screw clips that clip onto the drainage screw. For example, the drainage screw clip can be configured to releasably couple with a coupling groove on the drainage screw. The coupling groove on the screw can also be a cap groove.

Each of the drainage screw clips can have a clip feature configured to couple with the drainage screw during implantation and then release the screw after being sufficiently implanted. The clip feature can be flexibly resilient so as to be capable of having an inwardly biased orientation to hold the screw, and the clip feature can be biased outwardly to release the screw.

The drainage screw release member can have a lumen configured for being slidably received over a shaft of the drainage screwdriver. The lumen can be within a collar that is adapted to slide along the screwdriver. As such, the release member can have a collar with a lumen and luminal surface configured to slide with respect to the drainage screwdriver. The luminal surface can be a slidable surface as described herein and with or without a lubricious coating.

Optionally, the drainage screw release member has a release mechanism. The mechanism can be actuated or activated for releasing the drainage screw from the drainage screwdriver. The release mechanism can allow for the actuation of the mechanism to activate the release system and/or clip features so that the drainage screw is disengaged from the drainage screwdriver so that withdrawal of the screwdriver away from the screw can result in implantation of the screw in a tissue. The release mechanism can also be activated to change drainage screws on a drainage screwdriver.

In another option, the drainage screwdriver shaft can include a stop member that is configured to inhibit the distal motion of the release system and/or collar. The stop member can be a nodule, protrusion, or other feature that inhibits distal motion. For example, the stop member can be similar to the engaging members that are located on the shaft. A stop member may also be positioned to stop proximal motion of the release system and/or collar. Accordingly, the stop member can be configured to inhibit longitudinal motion of the drainage screw release member.

The drainage screw has an internal lumen that is smooth and configured to slide over the shaft of the drainage screwdriver, and configured as a drainage tube so as to be capable of draining a cyst or abscess when implanted therein. The internal lumen is defined by a luminal surface that is configured to have a cross-sectional profile that matches the cross-sectional profile of the drainage screwdriver shaft.

The luminal surface of the drainage screw includes one or more engaging features configured to engage with the drainage screwdriver. The engaging features are configured to be engagedly received with one or more drainage screw engaging members of the drainage screwdriver. The engagement of the engagement features with the drainage screw engaging members is sufficient to permit rotation of the drainage screw upon rotation of the drainage screwdriver and apply pressure to the drainage screw when pressure is applied to the drainage screwdriver. The engaging features can be recesses when the engaging members are protrusions, and can be protrusions when the engaging members are recesses. Alternatively, the engaging features can be a roughened surface that friction fits a roughed surface (e.g., engaging members) of the shaft.

The drainage screw can include a threaded external surface that allows the drainage screw to be screwed into a tissue. The threaded external surface can be configured as any type of threading that is found on any type of screw. The threads of the threaded external surface can be common threads or can be configured as blade threads, which can be clockwise or counter-clockwise.

The drainage screw has a tissue stop that has a larger cross-sectional profile compared to a screw surface of the drainage screw. The tissue stop is configured to inhibit the screw from penetrating further into the tissue. Once the screw is received into a tissue up to the tissue stop, the screw can be released from the drainage screwdriver. Accordingly, the tissue stop has a cross-sectional profile of a sufficient size so as to inhibit penetration into a tissue of a subject upon screwing the drainage screw into the tissue with the drainage screwdriver. The tissue stop has a stop surface configured for placement adjacent to the tissue, and may be flat concave, or convex. The tissue stop surface is from 45 degrees to about 90 degrees from the screw surface, either in acute or oblique orientation.

The drainage screw can include a drainage cap receiving feature that is configured to receive a drainage cap. Particularly, the drainage cap receiving feature is configured to removably receive the drainage cap. In one aspect, the drainage cap receiving feature is configured as a groove about a perimeter of the drainage screw. The groove may also be used to receive the clip features of the release member. In another aspect, the drainage cap receiving feature can be threads to match threads on the drainage cap. The drainage cap receiving feature may also include a lip or annular protrusion configured to retain the drainage cap on the drainage screw. In another aspect, the drainage screw can include a fastening member configured to fasten with the drainage cap to the screw. The fastening member can be any type of fastening member, which may include the features described herein.

The drainage cap has a cap body that is configured to be compatible with the screw, and can include a substantially flat or planar body similar to many different types of caps. The cap body can be rigid or elastic. The cap body can include an outer perimeter being a cap ring. The cap ring can be rigidly flexible so as to be similar to an elastic. For example, the cap ring can be configured as an o-ring coupled to the cap body.

In one option, the drainage cap and drainage screw are configured with threads for the drainage cap to screw onto the drainage screw.

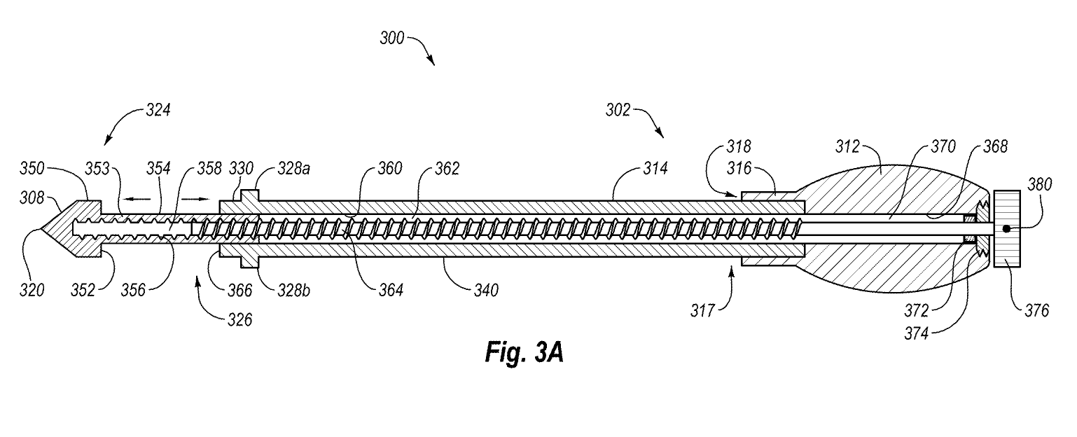

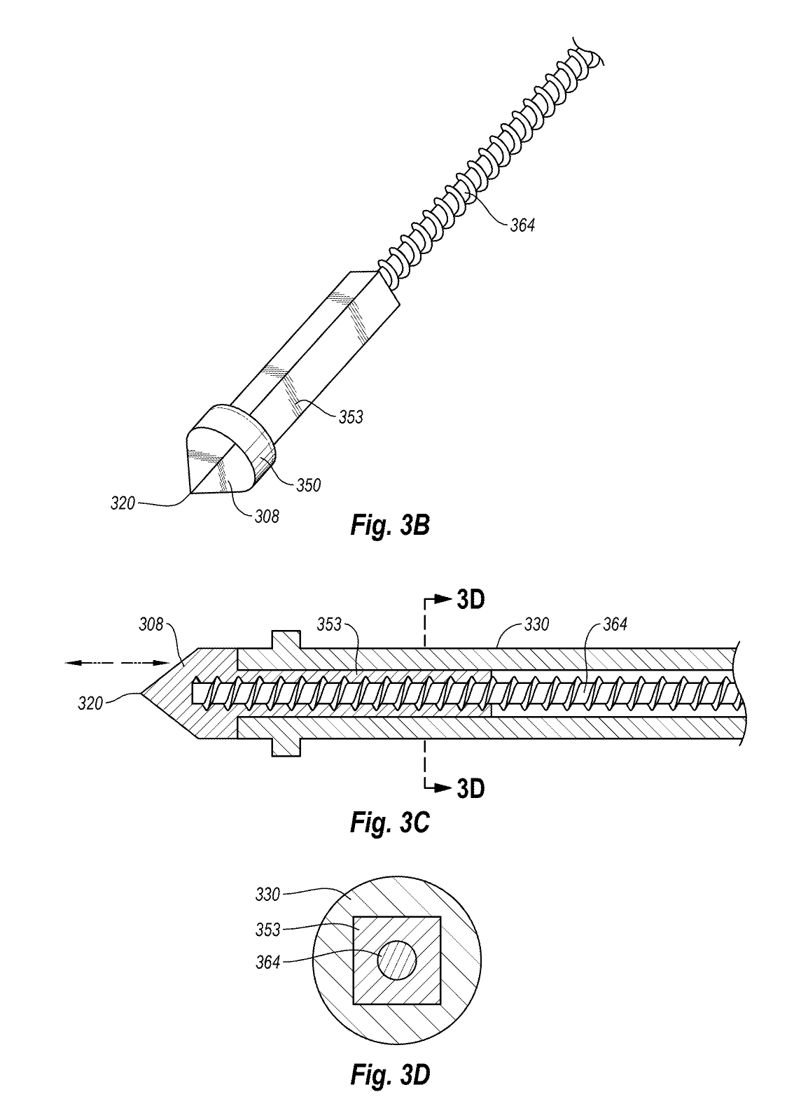

FIGS. 3A-3C show a fenestration system 300 that includes a mechanically elongatable/retractable screwdriver 302 that is configured for retaining a drainage screw 104 as shown in FIG. 1A. As such, the mechanically elongatable/retractable screwdriver 302 can retain the drainage screw 104 in a manner that allows for the mechanically elongating screwdriver 302 to screw the drainage screw 104 into a tissue. The mechanically elongatable/retractable screwdriver 302 includes a distal tip 308 that is sharp and configured for piercing a tissue. An elongatable/retractable shaft 314 couples the distal tip 308 to an opposite handle 312. The mechanically elongatable/retractable screwdriver 302 can also be configured to include a release member 106, as shown in FIG. 1A, which is slidably coupled with the elongating shaft 314 as shown by the arrows.

FIG. 1B-1D show the elongatable/retractable head 350 having a square cross-sectional profile; however, the cross-sectional profile can be any shape except for round because there needs to be one or more surfaces 354 that engage with the inner surface 360 of the elongatable/retractable shaft 314. FIG. 1D is a cross-sectional diagram of FIG. 1C as shown. Examples of cross-sectional profiles for the elongatable/retractable head 350 include triangle, square, rectangle, pentagon, hexagon, or other polygon.

The mechanically elongatable/retractable screwdriver 302 is shown without the drainage screw 104. As shown, the mechanically elongatable/retractable screwdriver 302 includes a distal tip 308 coupled to an elongatable/retractable shaft 314, which in turn is coupled to the handle 312. The distal tip 308 is included at a tissue piercing expandable/retractable end 324 of the mechanically elongatable/retractable screwdriver 302 that has a sharp tip 320 that is sufficiently sharp for penetrating a tissue upon contact and application of slight pressure.

The distal portion 326 (e.g., drainage screw receiving segment) of the elongatable/retractable shaft 314 is configured with an elongatable/retractable surface 330 that expands to pierce the tissue and still allows for the drainage screw 104 to slide onto and off from the elongating shaft 314. The elongatable/retractable surface 330 can extend from the distal tip 308 to the handle 312; however, the elongatable/retractable shaft 314 can include one or more screw engaging members 328a-328b. These screw engaging members 328a-328b are configured for engaging with the drainage screw 104 so that rotation of the mechanically elongatable/retractable screwdriver 302 rotates the drainage screw 104 and pressure applied to the screwdriver is transferred to the drainage screw 104. While the engaging members 328a-328b are shown as protrusions from the elongatable/retractable shaft 314, they can be indentations, a roughened surface, or the like. The engaging members 328a-328b can have substantially any shape that allows for the described functionality. When implemented as protrusions, the engaging members 328a-328b can be integrated with the elongatable/retractable shaft 314 or received into slots (note shown) in the elongatable/retractable shaft 314.

The elongatable/retractable end 324 includes an elongatable/retractable head 350 that is rotatably coupled to a screw shaft 364. As such, the elongatable/retractable head 350 includes a hollow shaft 353 that has an outer surface 354 and an inner threaded surface 356 that defines an inner lumen 358. The inner threaded surface 356 is rotatably coupled with the screw shaft 364, such that rotation of the screw shaft 364 in a first rotation direction propels the expandable head distally (e.g., expansion), and rotation in a second rotation direction propels the expandable head proximally (e.g., retraction). The elongatable/retractable head 350 is configured such that the distal tip 308 is associated with a lateral surface 352 that couples the distal tip 308 with the outer surface 354. The elongatable/retractable shaft 314 includes a lateral surface 366 that corresponds with the lateral surface 352, such that a first rotation direction of the screw shaft 364 pushes the lateral surfaces 352, 366 apart (e.g., expansion), and rotation in the second direction pulls the lateral surfaces 352, 366 together (e.g., retraction).

The elongatable/retractable shaft 314 can include an outer surface 340 and an inner surface 360 defining an internal lumen 362. The screw shaft 364 is included within the internal lumen 362, and extends from being rotatably coupled to the elongatable/retractable head 350 to the handle 312.

The proximal end 317 of the elongatable/retractable shaft 314 can be coupled to the handle 312 by any way a standard screwdriver shaft is coupled to its handle. As shown, the proximal end 317 of the elongatable/retractable shaft 314 is received into a handle recess 318 within the handle 312 and a handle fastener 316 fastens the handle 312 to the elongatable/retractable shaft 314.

Within the handle 312, the screw shaft 364 extends within the handle recess 318 into an internal lumen 370 defined by an inner surface 368. At the proximal end of the internal lumen 370, the screw shaft 364 includes a gasket 372 that is configured for preventing the screw shaft 364 from moving backwards. Also, a limit screw 374 is located proximally from the gasket 372, which also prevents the screw shaft 364 from moving backwards. The proximal end of the screw shaft 364 is coupled to a screw knob 376, where rotation of the screw knob 376 rotates the screw shaft 364. The screw shaft 364 can be coupled to the screw knob 376 by any functional fastener that allows for rotation of the screw knob 376 to rotate the screw shaft 364. For example, as shown the screw knob 376 includes a fastener screw 380 extending therethrough to the screw shaft 364 such that the fastener screw 380 engages the screw shaft 364. Thus, rotation of the screw knob 374 rotates the screw shaft 364 and either expands the elongatable/retractable head 350 or retracts the expandable head.

Also, the mechanically elongatable/retractable screwdriver 302 can be used to deliver a drainage screw 104 without the release member. The mechanically elongatable/retractable screwdriver 302 can be in an elongated orientation, as shown in FIG. 3A, and receive the drainage screw 104 thereon so that it engages with the engaging members 238a, 328b such that the sharp tip 320 extends past the drainage screw 104. The mechanically elongatable/retractable screwdriver 302 can then be used to pierce tissue and screw into the tissue by rotating the handle 312 which correspondingly rotates the drainage screw 104. After the drainage screw 104 is sufficiently inserted into the tissue, the screw knob 376 can be rotated so that the expandable/retractable head 350 retracts into the lumen of the drainage screw 104. The expandable/retractable head 350 can then be withdrawn from the drainage screw 104, which implants the drainage screw 104 into the tissue.

Additionally, the mechanically elongatable/retractable screwdriver 302 can retain the drainage screw 104, and can be placed at a tissue. Rotation of the screw knob 376 can expand the expandable/retractable head 350 so as to pierce the tissue, and then the handle 312 can be rotated to screw the drainage screw 104 into the tissue. The screw knob 376 can then be rotated the opposite direction so as to retract the expandable/retractable head 350 into the lumen of the drainage screw 104, and the mechanically elongatable/retractable screwdriver 302 can be withdrawn from the drainage screw 104 as described. Also, the mechanically elongatable/retractable screwdriver 302 can be used in the methods described below. Furthermore, the mechanically elongatable/retractable screwdriver 302 can be used in methods that include steps recited with regard to the operation of the mechanically elongatable/retractable screwdriver 302 as well as the steps recited below.

The embodiment of the mechanically elongatable/retractable screwdriver 302 can be used with different lengths of drainage screws. Since, the length is adjustable; it can be used for different lengths of screws for different types of tissues, tissue thicknesses, as well as cysts. Also, due to the existence of the inferior alveolar nerve in the center of the lower jaw, the device will not damage the nerve. Also, the mechanically elongatable/retractable screwdriver 302 can cause “a sense of falling” to be generated when the tip penetrates the bone cortex, and at that time, the screw knob 376 can be rotated such that the distal tip 308 is retracted into the drainage screw. Then the handle of the mechanically elongatable/retractable screwdriver 302 can be rotated so that only the drainage screw is engaged in the marrow cavity. The drainage screw end is blunt, and thereby the possibility of neural damage is greatly reduced with the mechanically elongatable/retractable screwdriver 302.

### Fenestration Methods

As described, the fenestration system can be useful for forming a drainage hole in a body tissue, such as a cyst or abscess sac. The fenestration system may also be used in other tissues as needed. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

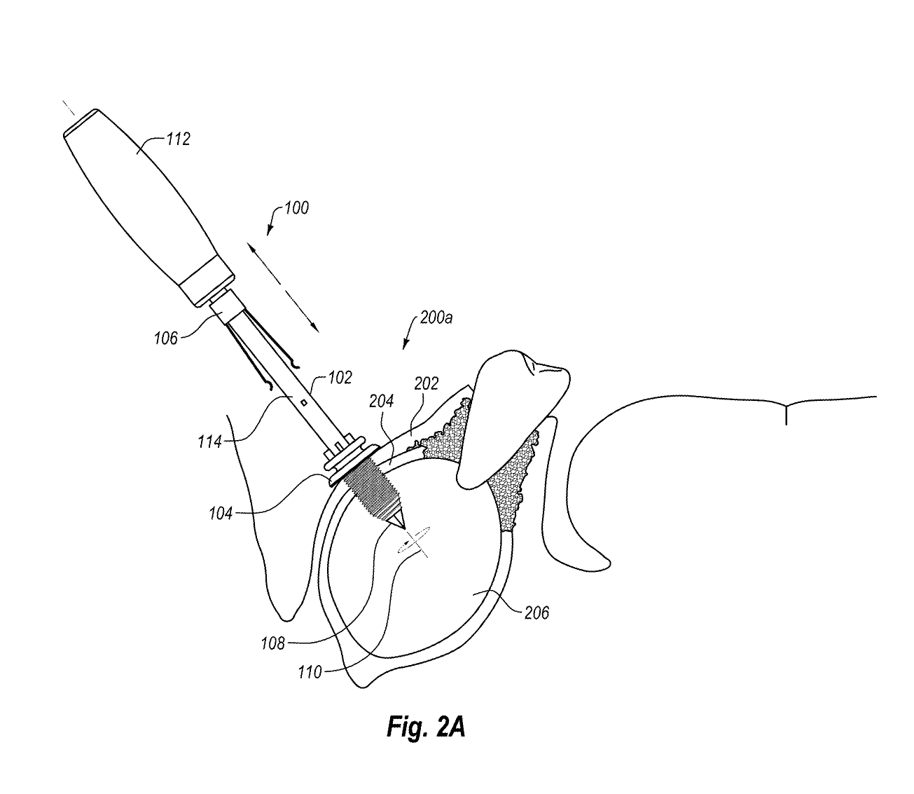

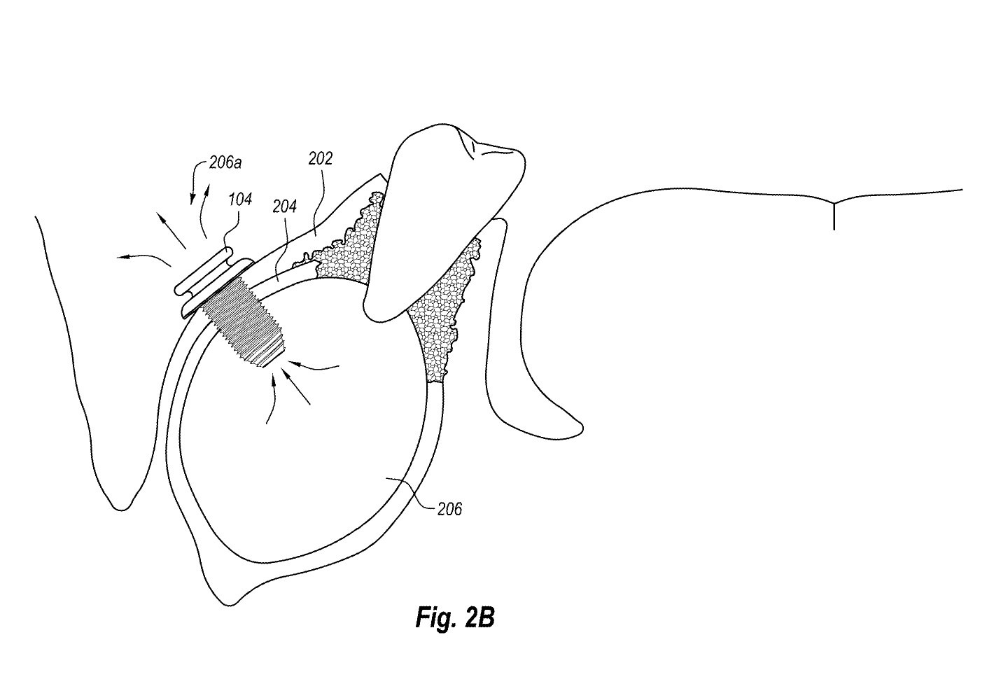

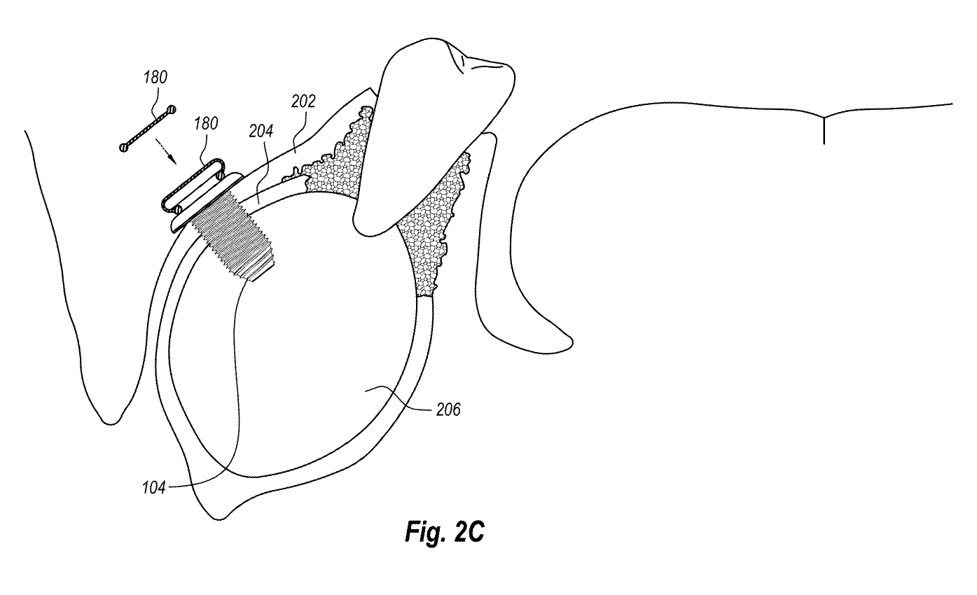

FIG. 2A shows a method (200a) of using a fenestration system 100 for inserting the fenestration screw 104 (i.e., drainage screw) into a cyst 206. FIG. 2B shows the drainage screw 104 functioning as a drainage tube and draining cyst contents 206a) from the cyst 206. FIG. 2C shows the screw cap 180 being applied to the drainage screw 104 so as to closure the internal lumen (not shown) of the drainage screw 104. The method (200a) can be performed by piercing the tissue 202 with the sharp distal tip 108 and then rotating the drainage screwdriver 102 around the axis of rotation 110. Such rotation screws the drainage screw 104 through the tissue 202, cyst sac 204, and into the cyst 206. As shown in FIG. 2A, the release mechanism 106 has been actuated such that the drainage screw 104 has been released and the shaft 114 can be withdrawn from the lumen (not shown) of the drainage screw 104. After a sufficient amount of drainage (shown in FIG. 2B) or before consuming a meal or at any other desired time, the screw cap 180 can be applied to the drainage screw 104 as shown in FIG. 2C to close the lumen of the drainage screw 104 and inhibit cyst contents 206a from passing out from the drainage screw 104 and also inhibits contaminants from entering the cyst 206 via the drainage screw 104.

In one embodiment, a method of draining a fluid from a subject can include: piercing a tissue associated with the fluid with a tip of a drainage screwdriver; driving a drainage screw into the tissue using a drainage screwdriver that is removably coupled to the drainage screw such that the drainage screw is inserted through the tissue and contacts the fluid; and uncoupling the drainage screwdriver from the drainage screw such that the drainage screw is retained in the tissue, and wherein an internal lumen of the drainage screw drains the fluid from the tissue.

In one embodiment, the drainage method can include sliding the drainage screw over a shaft and tip of the drainage screwdriver by withdrawing the shaft and tip from a lumen of the drainage screw. The drainage screw can then be disengaged from an engaging member of the drainage screwdriver after implantation into a tissue, which can allow for the shaft to be withdrawn from the drainage screw.

In one embodiment, the drainage screwdriver can include a release member can be activated in order to release the drainage screw from the drainage screwdriver. The type of release member can determine the type of activation, which can range from simply sliding the release member proximally or away from the drainage screw to activating a mechanism that releases the drainage screw, or manually decoupling the drainage screw from the release member. As such, the release of the drainage screw from the drainage screwdriver can include decoupling the drainage screw from the drainage screw release member. Such decoupling can include releasing a drainage screw clip from the drainage screw by activating a release mechanism. Also, the decoupling can include releasing the drainage screw clip from a groove in the drainage screw.

The ability of the drainage screw to be coupled to the drainage screwdriver allows for different types of drainage screws to be configured for different uses, such as for cysts or abscesses in the mouth as well as for cysts or abscesses in other parts of the body. Accordingly, a particular drainage screw (e.g., dimension) can be selected for the anatomy to be drained. When coupling a drainage screw to a drainage screwdriver the internal luminal surface of the drainage screw can be engaged with one or more engaging members of the drainage screwdriver.

In one embodiment, the drainage method includes piercing and/or driving the drainage screwdriver into the tissue. Such piercing and/or driving can include rotating the drainage screwdriver and screwing the drainage screw through the tissue. The handle of the drainage screwdriver can be sufficiently gripped by a practitioner so that force can be applied to facilitate the piercing and driving.

In one embodiment, the drainage screw can be driven into the tissue until the tissue contacts a tissue stop of the drainage screw. The tissue stop can have a larger cross-sectional profile compared to a screw surface of the drainage screw.

After the drainage screw is sufficiently implanted, the internal lumen of the drainage screw can be sealed or capped with a screw cap. This can include placing a screw cap over an opening of the internal lumen of drainage screw.

In one embodiment, the screw cap can include a flexible member that is stretched to fit over the drainage screw. For example, at least a portion of the flexible member can be configured as an o-ring.

Alternatively, the screw cap and drainage screw can have mating threaded members. As such, application of the screw cap to the drainage screw can include threading the cap with the drainage screw.

While the drainage screw is implanted into the tissue, the screw cap can be removed to permit additional drainage, and then re-applied to the drainage screw as desired. For example, the screw cap can be applied to the drainage screw before a meal, and then removed after a meal to permit additional drainage.

In one embodiment, the fenestration system can include a drainage tube that can be coupled to the drainage screw. The drainage tube can be coupled and released from the drainage screw similarly as described in connection to the screw cap. As such, the drainage tube can be mated to the internal lumen of the drainage screw to facilitate drainage. When the cyst or abscess is in the mouth, the drainage screw can be of a sufficient length to extend out of the mouth. This can allow for the cyst or abscess contents to drain out of the mouth rather than into it.

In one embodiment, the fenestration system can be used in a method of draining an oral cyst or abscess. Such a method can include: piercing a cyst or abscess sac in a jaw tissue with a sharp tip of a drainage screwdriver; driving the drainage screwdriver such that a drainage screw is inserted through the sac and contacts body fluid located therein; removing the drainage screwdriver from the drainage screw such that the drainage screw is retained in the sac with an internal lumen of the drainage screw draining the cyst fluid from the cyst sack; and applying a drainage cap to the drainage screw so as to seal the internal lumen of the drainage screw.

### Manufacturing

The screwdriver elongate shaft and shaft features can be metal, such as stainless steel, aluminum, galvanized steel, titanium, or other commonly used material for surgical and/or dental instruments such as ceramics or composite materials. Examples of metals can include stainless steel, silver, platinum, tantalum, palladium, cobalt-chromium alloys, or other known biocompatible metal materials. The distal end and handle may also be prepared from the same or different metal as well as from ceramic or composite material. Also, the handle may be formed from polymeric material, and may not be biocompatible material.

Examples of some ceramic materials that can be prepared into a screwdriver elongate shaft and shaft features can include but are not limited to aluminum oxides, zirconia, carbides, borides, nitrides, or silicides. Examples of composites can include but are not limited to woven wire with fiber metal felt and composite materials formed from polymers, metals, and/or ceramics.

The drainage screw can be prepared from various materials, such as but not limited to metals and alloys, polymers, biostable polymers, biodegradable polymers, ceramics, and composites. Examples of such as polymers can include but not limited to polyvinylidene difluoride (PVDF), polyethylene, polypropylene, polytetrafluoroethylene (PTFE), or polycarbonates. The drainage screw can also be prepared from the materials recited herein with respect to the screwdriver.

The screw can include a coating of, or be made from, biodegradable or bioabsorbable materials. The screw can be prepared from or coated with a biocompatible polymer. Examples of such biocompatible polymers can include a suitable hydrogel, hydrophilic polymer, biodegradable polymers, bioabsorbable polymers. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, combinations thereof, or the like.

Furthermore, the screw and/or screwdriver shaft can be formed from a ceramic material. In one aspect, the ceramic can be a biocompatible ceramic. Examples of suitable ceramic materials include hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like.

Moreover, the screw can include a radiopaque material to increase visibility using a machine. Radiopaque materials associated with implants can be viewed by a variety of techniques. Optionally, the radiopaque material can be a layer or coating any portion of the screw. The radiopaque materials can be platinum, tungsten, silver, stainless steel, gold, tantalum, bismuth, barium sulfate, or a similar material.

It is further contemplated that the external surface and/or internal surface of the screw can be coated with another material having a composition different from the primary implant material. The use of a different material to coat the surfaces can be beneficial for imparting additional properties to the screw, such as providing radiopaque characteristics, drug-reservoirs, and improved biocompatibility.

The coatings can also be provided on the screw to facilitate the loading or delivery of beneficial agents or drugs, such as therapeutic agents, pharmaceuticals and radiation therapies.

The drug or beneficial agent can be useful to improve the use of the drainage screw. Such drugs or beneficial agents can include antithrombotics, anticoagulants, antiplatelet agents, thrombolytics, antiproliferatives, anti-inflammatories, agents that inhibit hyperplasia, inhibitors of smooth muscle proliferation, antibiotics, growth factor inhibitors, or cell adhesion inhibitors, as well as antineoplastics, antimitotics, antifibrins, antioxidants, agents that promote endothelial cell recovery, antiallergic substances, radiopaque agents, viral vectors having beneficial genes, genes, siRNA, antisense compounds, oligionucleotides, cell permeation enhancers, and combinations thereof. Any beneficial agent can be used.

The cap can either be a resiliently deformable material that can be stretched to be applied to and removed from the drainage screw. Otherwise, the cap can be rigid and similar to any common bottle cap. When resiliently deformable, the cap can be prepared from an elastomer material.

An elastomer is a polymer with the property of viscoelasticity (“elasticity”), generally having notably low Young's modulus and high yield strain compared with other materials. The term, which is derived from elastic polymer, is often used interchangeably with the term rubber, although the latter is preferred when referring to vulcanisates. Each of the monomers which link to form the polymer is usually made of carbon, hydrogen, oxygen and/or silicon. Elastomers are amorphous polymers existing above their glass transition temperature, so that considerable segmental motion is possible. At ambient temperatures rubbers are thus relatively soft (˜3 MPa) and deformable. Examples of elastomers can include: Natural Rubber (NR); Synthetic Polyisoprene (IR); Butyl rubber (copolymer of isobutylene and isoprene, IIR); Halogenated butyl rubbers (Chloro Butyl Rubber: CIIR; Bromo Butyl Rubber: BIIR); Polybutadiene (BR); Styrene-butadiene Rubber (copolymer of polystyrene and polybutadiene, SBR); Nitrile Rubber (copolymer of polybutadiene and acrylonitrile, NBR), also called Buna N rubbers; Hydrogenated Nitrile Rubbers (HNBR) Therban and Zetpol; Chloroprene Rubber (CR), polychloroprene, Neoprene, Baypren; or other unsaturated rubbers. Examples of saturated rubbers that cannot be cured by sulfur vulcanization can include: EPM (ethylene propylene rubber, a copolymer of ethylene and propylene) and EPDM rubber (ethylene propylene diene rubber, a terpolymer of ethylene, propylene and a diene-component); Epichlorohydrin rubber (ECO); Polyacrylic rubber (ACM, ABR); Silicone rubber (SI, Q, VMQ); Fluorosilicone Rubber (FVMQ); Fluoroelastomers (FKM, and FEPM) Viton, Tecnoflon, Fluorel, Aflas and Dai-El; Perfluoroelastomers (FFKM) Tecnoflon PFR, Kalrez, Chemraz, Perlast; Polyether Block Amides (PEBA); Chlorosulfonated Polyethylene (CSM), (Hypalon); Ethylene-vinyl acetate (EVA); or others. Other elastomers can include: Thermoplastic elastomers (TPE), for example Elastron; Thermoplastic Vulcanizates (TPV), for example Santoprene TPV; Thermoplastic Polyurethane (TPU); Thermoplastic Olefins (TPO); the proteins resilin and elastin; Polysulfide Rubber; or others.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

1. A fenestration system comprising:
a drainage screwdriver;
a drainage screw removably receivable onto the drainage screwdriver; and
a drainage cap removably coupleable with the drainage screw when the drainage screw is removed from the drainage screwdriver.

2. The fenestration system of claim 1, wherein the drainage screwdriver includes one or more of:
a shaft with a smooth drainage screw receiving surface configured to receive the drainage screw thereover, and the shaft includes one or more drainage screw engaging members;
a handle coupled to a screwdriver shaft or
a shaft with a tissue piercing tip.

3. The fenestration system of claim 1, wherein the drainage screw has an internal lumen defined by a luminal surface configured to slide with respect to a screw-receiving surface of the drainage screwdriver, and includes one or more engaging features configured to engage with the drainage screwdriver.

4. The fenestration system of claim 1, wherein the drainage screw includes a screw surface that has threads.

5. The fenestration system of claim of claim 1, wherein the drainage screw includes a drainage cap receiving feature configured to removably receive the drainage cap.

6. The fenestration system of claim 1, wherein the drainage cap has a cap body with an outer perimeter being a flexible cap ring.

7. The fenestration system of claim 1, wherein the drainage screwdriver includes a drainage screw release member with a drainage screw clip configured to releasably couple with a coupling groove on the drainage screw.

8. The fenestration system of claim 7, wherein the drainage screw release member has a collar with an internal lumen configured for being slidably received over a shaft of the drainage screwdriver.

9. A method of draining a fluid from a subject, the method comprising:
piercing a tissue associated with the fluid with a tip of a drainage screwdriver;
driving a drainage screw into the tissue using the drainage screwdriver removably coupled to the drainage screw such that the drainage screw is inserted through the tissue and contacts the fluid; and
uncoupling the drainage screwdriver from the drainage screw such that the drainage screw is retained in the tissue, and wherein an internal lumen of the drainage screw drains the fluid from the tissue.

10. The method of claim 9, wherein uncoupling the drainage screwdriver from the drainage screw includes one or more of the following:
sliding the drainage screw over a shaft and tip of the drainage screwdriver;
releasing the drainage screw from an engaging member of the drainage screwdriver;
activating a drainage screw release member of the drainage screwdriver to release the drainage screw from the drainage screwdriver;
releasing a drainage screw clip from the drainage screw by activating a release mechanism;
releasing the drainage screw clip from a groove in the drainage screw; or
sliding the drainage screw release member away from the drainage screw after activating a drainage screw release member.

11. The method of claim 9, further comprising withdrawing the drainage screwdriver from a lumen of the drainage screw after the drainage screw is placed in the tissue.

12. The method of claim 9, further comprising coupling the drainage screw to the drainage screwdriver by engaging an internal luminal surface of the drainage screw with one or more engaging members of the drainage screwdriver.

13. The method of claim 9, wherein the piercing and/or driving includes one or more of the following:
rotating the drainage screwdriver;
screwing the drainage screw through the tissue;
griping a handle of the drainage screwdriver; or
driving the drainage screwdriver into the tissue until the tissue contacts a screw stop of the drainage screw.

14. The method of claim 9, further comprising sealing the internal lumen of the drainage screw by placing a cap over an opening of the internal lumen of drainage screw.

15. The method of claim 14, further comprising removing the cap to permit additional drainage.

16. The method of claim 9, wherein the tissue comprises tissue includes a cyst or tissue surrounding a cyst located within a mouth of a subject.

17. The method of claim 9, further comprising:
piercing a cyst sac in a jaw tissue with a sharp tip of a drainage screwdriver;
driving the drainage screwdriver such that a drainage screw coupled with the drainage screwdriver is inserted through the cyst sac and contacts cyst fluid located therein; and
decoupling the drainage screwdriver from the drainage screw such that the drainage screw is retained in the cyst sac with an internal lumen of the drainage screw draining the cyst fluid from the cyst sac.

18. The method of claim 9, wherein the fenestration screwdriver includes:
an elongate shaft having drainage screw engaging members configured to receive a drainage screw;
a sharpened tip at one end of the elongate shaft;
a handle at one end of the elongate shaft opposite of the sharp tip; and
a drainage screw release system configured to retain the drainage screw during implantation and to release the drainage screw from the elongate shaft after implantation.

19. The method of claim 9, wherein the fenestration screw includes:
a tubular body having an outer surface and an inner surface, the outer surface including:
a distal end having a first opening to an internal lumen;
a screw section proximal to the distal end and including screw threads;
a stop proximal to the screw section and having a larger cross-sectional profile compared to the screw section and configured to inhibit the fenestration screw from advancing further into a tissue past the stop;
a releasable member proximal to the stop and being configured to be coupled to a release system during implantation and be released from the release system after implantation, wherein the releasable member is also configured to removably receive a screw cap; and
a proximal end having a second opening to the internal lumen; and
the inner surface including:
a smooth luminal surface proximal of the first opening and defining the internal lumen; and
one or more engagement members formed into the smooth luminal surface and configured to engage with a fenestration screwdriver.

20. A fenestration screwdriver comprising:
an elongate shaft having an internal lumen defined by an inner surface;
one or more engaging members located on an outer surface of the elongate shaft, wherein the engaging members are configured to engage with the drainage screw;
a screw shaft located within the internal lumen of the elongate shaft such that a distal end and a proximal end each extend from the internal lumen;
an elongatable/retractable member having a sharp tip and an outer surface movably coupled with the inner surface of the elongate shaft so as to be at least partially located within the internal lumen of the elongate shaft and having an threaded inner surface defining an inner lumen of the elongatable/retractable member, the threaded inner surface is rotatably coupled with the distal end screw shaft that such that the screw shaft is at least partially located within the internal lumen of the elongatable/retractable member;
a handle having an internal lumen defined by an inner surface, the proximal end of the screw shaft extending through the internal lumen of the handle; and
a screw knob coupled to the screw shaft such that rotation of the screw knob rotates the screw shaft.
